(19) United States
(12) Patent Application Publication
(10) Pub. No.: US 2014/0188229 A1
(43) Pub. Date: Jul. 3, 2014

(54) Implantable biological joint replacement

(71) Applicant: Bernhard HILDEBRANDT, Bad Wildbad (DE)

(72) Inventor: Bernhard HILDEBRANDT, Bad Wildbad (DE)

(21) Appl. No.: 14/066,051

(22) Filed: Oct. 29, 2013

Related U.S. Application Data
(63) Continuation-in-part of application No. PCT/DE2012/000415, filed on Apr. 17, 2012.

(30) Foreign Application Priority Data
Apr. 29, 2011 (DE) 10 2011 100 073.2
Jun. 3, 2011 (DE) 10 2011 103 457.2

(51) Int. Cl. A61F 2/30

(52) U.S. Cl. CPC A61F 2/30756
USPC 623/18.11

(57) ABSTRACT

The present invention concerns the problem of solving issues of common mechanical endoprostheses and limitations of current cartilage replacement methods by manufacturing a complete implantable biological joint replacement. Devices which provide for the shaping, diffusion activation and particularly fulling of the joint replacement which is being produced—i.e. compression with alternating pressure and possibly a gliding motion of the fulling device on the surface of the joint replacement—are used in order to produce a weight bearing biological joint replacement with hyaline cartilage. Substances and processes which activate the formation of hyaline cartilage tissues are also provided. At the present time, the biological joint replacement is preferably suitable for implantation into the shoulder and hip, but can also be used for other joints by applying multidimensional grinding techniques.

### FIELD OF THE INVENTION

The invention concerns a device which is used in culturing at least a component of a biological joint replacement, and specifically, activates its differentiation into a joint replacement which is capable of bearing weight; in that a fulling point, that is, a maximum pressure range—or in the case of hinge joints, a fulling line—repeatedly progresses largely over the entire joint replacement surface by a moving fulling bowl, whereby the fulling bowl at least possesses a shape which comes close to that of the joint replacement surface, and flushes joint replacement production substances into the fulling gap which is produced in this manner.

### STATE OF THE ART

The worldwide number of arthroses (“joint wear”) is increasing. Sooner or later, they can only be corrected with a joint replacement. Current metal endoprostheses with gliding surfaces made from metal, ceramics or plastics only have a limited service life and weight bearing capability, aside from other problems.

At the present time, however, only minor cartilage defects can be replaced by inserting cartilage components which were taken from other locations or cultured. Arthroplastic abrasion is limited to arthroses which are not very advanced, and can only delay the problem to some degree. Culturing (tissue engineering) of tissues has made major advances. However, it was not possible thus far to produce a perfectly hyaline joint replacement which is capable of bearing weight, and therefore implantable.

### SUMMARY OF THE INVENTION

The present invention concerns the problem of solving issues of common mechanical endoprostheses and limitations of current cartilage replacement methods by manufacturing an implantable biological joint replacement.

This problem is solved by the devices with the attributes of claim 1. The sub-claims list various technical embodiments and joint replacement production substances 6a of the fulling device.

In order to produce a joint replacement with hyaline cartilage which can bear full weight, the process according to the invention is that in addition to the devices known in tissue engineering processes, it possesses a fulling device which provides the prerequisites for the growth and generation(=differentiation) of a joint replacement, particularly a cartilage layer which is capable of bearing weight, but also the carrying layer (preferably consisting of bone) and possibly also a connective layer. Herein a fulling process (fulling motion) is performed by the fulling device, at least mainly across the entire surface of the joint replacement being formed, by a fulling point or fulling line which moves over this surface. Also an additional gliding motion of the fulling bowl (or the joint replacement surface which is positioned on a fulling bowl) may be provided against the other joint replacement surface. Together with flushing at least one joint replacement production substance into the fulling gap by means of a flushing device—and the diffusion of these substances into the tissues, which is activated by the fulling process—this activates a specific tissue differentiation of the joint replacement or its parts, i.e. the formation of a biological joint replacement with the required weight bearing capacity.

The fulling device for producing a weight bearing biological joint replacement by means of a specific fulling point/fulling line motion which is performed by fulling bowls whose shape and curvature at least nearly corresponds to the joint replacement surfaces as described in the patent claims is not known, and research showed no indicators of similar patent applications.

Regarding cultivation procedures of tissues for materials which are suitable for joints, there are e.g. the following applications:

| | | |
|---|---|---|
| DE 199 26 083 A1 | DE 38 10 803 A1 | DE 195 43 110 A1 |
| DE 197 21 661 A1 | EP 0 339 607 A2 | EP 0 530 804 A1 |
| EP 0 739 631 A2 | WO 96/03160 A1 | WO 97/46665 A1 |
| WO 98/42389 A1 | WO 99/25396 A2 | |

The following applications

| | | |
|---|---|---|
| US 2002/106625 A1 | WO 2010/005917 A2 | WO 2009/011849 A2 |
| WO 02/48317 A2 | DE 101 04 008 A1 | |

also show compression of the tissues being cultured, usually also with varying pressure loads, and US 2002/106625 A1 also with only partial pressure loads on the surfaces. WO 2010/005917 A2 also specifies rollers which roll over the tissue being produced in a flat plane, i.e. they are not usable for a shaped joint replacement.

These rollers fundamentally differ from the fulling bowls of the invention and do not have the specific effects of the fulling device according to the invention, since the curvature of the roller deviates far from that of the tissue surface, and thereby the pressure acts on a relatively small surface and results in strong lateral compression shifting of the adjacent tissues with its cells, which—without destroying them—only allows a pressure load which is not sufficient to activate the tissues into a weight bearing differentiation, also partly because the joint replacement production substances between the roller and the joint surface are mainly shifted by the roller, and not strongly activated to diffuse into the tissues; which, however, does occur in the fulling process according to the invention. None of these patent applications features a fulling device which corresponds to that of the invention.

Further benefits result from the sub-claims and the following description.

### BRIEF DESCRIPTION OF THE FIGURES

The invention is described in further detail in the following, using exemplary embodiments which are shown in the included figures.

FIG. 1 shows the principle of the invention, i.e. the fulling device with fulling bowls, the fulling gap, the fulling point and their fulling pressure effects, the fulling motion and its influence on the tissues being cultured, as well as the joint replacement production substances which are flushed into it, particularly also the difference existing therein as compared to known tissue culturing devices;

FIG. 2-5 symbolically show the principle of drive and motion devices by which the fulling motion is transferred to the fulling bowls, and through these to the joint replacement;

FIG. 6-9 show the use of the joint replacement production substances in the fulling device by means of a flushing device and examples of the characteristics of the fulling devices with which the flushing into the joint gap and diffusion into the joint replacements is activated;

FIG. 10 shows a mechanical embodiment of the fulling device and an example in which both fulling bowls are occupied by joint replacements and fulling takes place from one joint replacement onto the other;

FIG. 11 shows an exemplary embodiment of a shape development fulling, i.e. the fulling process in which not only fulling is performed to differentiate the tissues, but also tissue cultivation, i.e. it is also shaped by the fulling device;

FIG. 12-14 show examples of mechanical fulling motion devices which can be used both in a ball joint and in a hinge joint, and in which specifically the fulling of a ball joint can be associated with a gliding motion;

FIG. 15 shows another example of a mechanical fulling device with two circular tracks which are mounted in planes vertically standing on each other, via which the fulling motion is performed;

FIG. 16-19 show examples of force transmission devices on the fulling bowls;

FIG. 20-21 show an example of a fulling device in which the fulling pressure generated by a pressure system is transferred to the fulling bowl via flexible pressure chambers or directly onto the joint replacement, whereby the pressure chambers completely cover the fulling bowl or joint replacement.

### DETAILED DESCRIPTION OF PREFERRED EXEMPLARY EMBODIMENTS

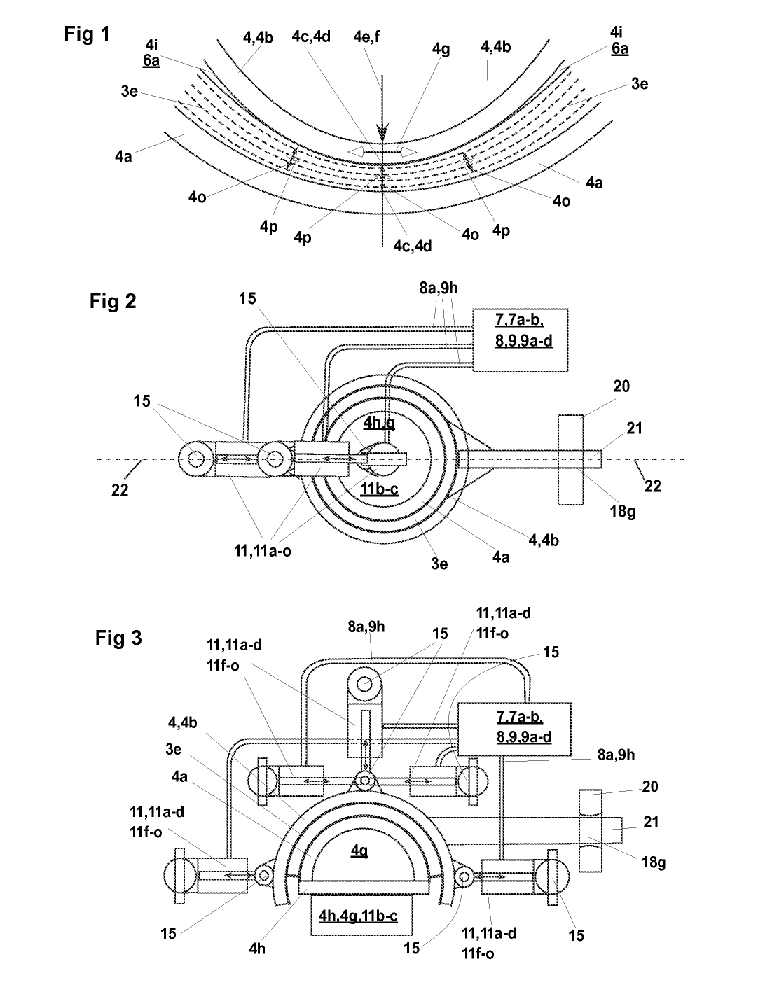

The Principle of the Fulling Device is Shown in Combination with FIG. 1:

FIG. 1A fulling bowl 4 (here joint bowl 4b) applies fulling pressure 4e or a fulling depth 4f on the other fulling bowl 4a (here base bowl 4a), namely on the joint side of joint replacement 3e, whereby the maximum pressure region is the fulling point 4c or the fulling line 4d, on which the minimum gap between the fulling bowls is usually also located. Since the shape of the fulling fulling bowl/joint bowl 4, 4b approximates the shape of joint replacement 3e, the fulling pressure force 4o on the tissues therefore only lessens to a moderate degree in the radial direction, deviating from fulling point 4c (or fulling line 4d). Consequently, squeezing apart—that is, laterally forcing the tissues away due to compression(=pressure crosswise to the fulling pressure 4p)—only results in a fraction of the pressure in the direction of the fulling pressure force 4o. In the course of this, the fulling point 4c (or the fulling line 4d) is repeatedly moved across approximately the entire surface of the joint replacement 3e, i.e. the surface of joint replacement 3e is fulled.

In some of the known processes, for instance, pressure is applied on cartilage with alternating pressure forces to produce the cartilage; however, this is done either by exposing its surface to consistent pressure, or by a pressure stamp exercising the same pressure on the surface, or by rolling a roller over the cartilage.

This, however, does not have the fulling effect according to the invention, since e.g. the roller applies pressure force on the cartilage which, due to the strongly differing curvature of the roller—e.g. as compared to the cartilage surface—squeezes the tissues which are being produced apart(=pressure force is crosswise to the pressure direction, meaning the tissues are squeezed to the sides), since this lateral pressure force is not met by nearly equally high pressure from the surrounding tissues and would therefore tear them if the pressure which were applied to them were sufficiently high to activate differentiation; that is, the cell walls and the tissue framework are exposed to excessive traction force if pressure which, in principle, is activated to differentiation by a roller crosswise in relation to its pressure force.

Due to these differences as compared to the device according to the invention, these pressure application processes in the thus far known devices for producing joint replacements or cartilage replacements are the reason why the differentiation of the cartilage tissues into a weight bearing joint replacement does not occur; that is, the processes which are thus far available could not be used to produce a biological joint replacement.

During the development of a joint, it is exposed to a pressure motion which is similar to that of the fulling device according to the invention.

In the invention, only a small fulling gap 4i is provided for differentiation fulling, with the specification, however, that there is not only joint lubricant in this narrow fulling gap 4i to produce the joint replacement 3a (as in the joint itself), but also at least one joint replacement production substance 6a, e.g. cells and/or tissue framework particles can also be flushed in. This is performed by a flushing device 6 (which is therefore provided in the inventive fulling device) through which the joint replacement production substance 6a is flushed into the fulling gap 4i from its edge or through inflow openings 10a, channels, holes, pass-through openings or porosity in at least one of the fulling bowls 4. For sealing to the outside, the fulling gap 4i can be provided with a fulling gap foil 4k, a fulling gap sealing bar 4l, a fulling gap foil pouch 4m, or a fulling gap sealing tube 4r.

The process of the fulling device in the production of the joint replacement 3e has a starting and end point, whereby the fulling device is used at least in the end area.

End area signifies that e.g. a cartilage layer 1 was cultured with one of the thus far available procedures, and the fulling device is then used to activate the differentiation into weight bearing cartilage 1(=differentiation fulling). Starting area means that even the beginning of the cartilage cultivation takes place under the influence of the fulling device, i.e. in the fulling device. In the course of this, fulling initially takes place without fulling pressure 4e(=pressure application), but only with a fulling depth 4f, i.e. a reduction of the gap between the fulling bowls 4, whereby the joint replacement tissues are cultured in this gap(=tissue culturing chamber 5=fulling gap 4i) by introducing cells 6c, tissue particles/framework particles/framework substance 6d. This can also be implemented by first producing a thin tissue layer whose thickness/strength then constantly increases, whereby the fulling depth 4f is adjusted to this increase by being reduced, and the fulling pressure 4e rises from practically zero at the outset to the end value.

This simultaneously shapes and differentiates the joint replacement 3e by the accordingly shaped fulling bowls 4, which may also be replaced by increasingly larger fulling bowls. In the course of this, e.g. cartilage or cartilage particles are placed in a shaping fulling device (see FIG. 11), and the fulling process is used to produce both the planned shape and the development of the weight bearing structures (e.g. spongiosa bars and hyaline cartilage). The particles may consist of particles of framework substance 6d and cells 6c or of tissue particles in which the framework substance and the cells are a tissue-specific unit. This shaping fulling process then increasingly changes to a differentiation fulling process.

In order to continue building the joint replacement 3e in the fulling device after commencing cultivation, the fulling gap 4i can reach a low width/gap at a certain distance from the fulling point 4c or the fulling line 4d, into which tissue particles (framework particles) 6d and cells 6e are flushed, or while the fulling bowl 4 is repeatedly lifted slightly away from the joint replacement surface during the fulling process (particularly if the fulling point 4c is located in the marginal areas of the joint which is being produced), and the fulling gap 4i thereby reaches a width/distance which is provided for the required process of flushing in the tissue particles (framework particles) 6d and cells 6e.

Overall, the joint replacement 3a can therefore be produced in one work step with the fulling device according to the invention, or it can take place in several processes as follows:
the application or introduction of new bone or cartilage particles takes place in several fulling processes, whereby the size of the fulling bowls 4 can be increased,
and/or subsequent fulling processes are performed with fulling bowls 4 in different shapes, fulling bowls 4 in different sizes, or fulling bowls 4 made from different materials, whereby this can also take place in different fulling devices,
and/or separate fulling processes (including shape forming fulling) are performed for different parts of the joint replacement 3a,
and/or fulling is performed to combine at least two of the layers 1 to 3 of the joint replacement 3a,
and/or fulling (including shaping fulling processes) is/are performed with additional or different fulling bowls 4 and/or joint gap mats 4n and/or fulling gap foil pouches 4m and/or with different joint replacement production substances 6a and/or joint lubricants 6e,
and/or at least one grinding/polishing process is performed after a fulling process as needed (including the shaping fulling process), i.e. fulling processes and grinding/polishing processes are also provided subsequently, as needed.

Furthermore, a gliding motion drive 4g is provided as needed; this causes a gliding motion of the joint bowls 4b or of a joint replacement 3e which is positioned on a fulling bowl 4 against the surface of the other joint replacement 3e.

In addition to the substances thus far used for cell cultivation, it is also possible to use e.g. joint lubricants 6e (or also substances which contribute to its development, such as synovial fluids, glucosamine, chondroitin, hyaluronic acid or collagen). These were not used in tissue engineering of joint replacements thus far. Even in the event that the fulling device according to the invention does not provide an additional gliding motion, a fulling process can result in a gliding motion—however minimal—particularly at the maximum distance to the fulling point 4c/the fulling line 4d. The use of joint lubricants (also with substances which are dissolved or dispersed in them) can also have favorable effects due to the compression at the fulling point 4c, particularly into the cartilage layer, and furthermore activate the differentiation, particularly of the cartilage layer, into hyaline, weight bearing cartilage. Aside from known differentiation and tissue engineering substances 6b, specific substances 6g can also be considered in this regard (e.g. methylsulfonylmethanes(=MSM), anti-interleukin-1 (IL 1-Ra)), which can have this effect when pressed into cartilage, bone and/or connective layers.

Differentiation substances 6f which activate tissue differentiation particularly in combination with fulling are also provided. Furthermore, connection substances 6h, which encourage the activation of a connection between two layers of the joint replacement 3e under the influence of the fulling motion and/or generate a connective layer on the side of the bearing layer 2, which is later applied to the bony joint head/joint pan, are also provided.

The following provides an overview and definition of the control devices 8 and motion devices 11, which generate the fulling motion with the fulling perform device 7 (that is, a fulling motion device 7a and a fulling intensity device 7b), and which are shown as examples in the following diagrams:

The control device 8 is combined with the fulling perform device 7 and controls at least the fulling intensity device 7b and/or the fulling motion device 7a and/or the flushing device 6, namely e.g. by means of electronic control devices, e.g. programmable and/or processor guided control devices, approximately corresponding to those in CNC devices, and it possesses at least one of the following setting devices for this purpose: mechanical, hydraulic, hydropneumatic, pneumatic or electrical setting devices.

The motion devices 11 are based on at least one of the following systems/devices:
a pressure system 9, and this, in turn, from a pneumatic pressure system 9a, hydraulic system 9b, hydropneumatic pressure system 9c or hydrodynamic pressure system 9d, whereby these transmit the fulling motion to the fulling bowl 4 via pressure lines 9h by means of e.g. pneumatic hydraulic 11j, hydropneumatic 11k, hydrodynamic motion devices 11l.
electric 11f, electromagnetic 11g, electromotor 11h, mechanical motion devices 11m (whereby these can consist e.g. of a longitudinal motion device and/or an elastic device to generate pressure), whereby the fulling perform device 7 may also possess a cooperation and/or combination of at least two of these devices, in which the motion devices 11 consist of e.g. linear 11a, pivoting 11b, rotation motion devices 11c and motion devices 11e which act into one direction 11d or into both directions.

FIG. 2-5 show symbolic examples of fulling perform devices 7 (for differentiation and shaping fulling processes), i.e. fulling motion devices 7a and fulling intensity devices 7b which are controlled by a control device 8 via control lines 8a. The motion devices act by force transmission 11n, e.g. force transmission rods 11o directly e.g. via radial joint bearings 15 or other connections on at least one of the fulling bowls 4 (that is, the base bowl 4a and/or the joint bowl 4b) and move them in such a manner that the fulling process, i.e. the motion of the fulling point 4c(=maximum pressure point) or the fulling line 4d(=maximum pressure line) occurs over the surface of the joint replacement 3e, which consists of at least one of the following layers: Cartilage layer 1, carrying layer 2, connective layer 3 between the cartilage and the carrying layer, i.e. the joint replacement 3e within the scope of the invention also includes the complete joint replacement 3a.

In order to achieve an additional gliding motion either between one fulling bowl 4 and the joint replacement 3e or between the joint replacement parts 3e which are mounted on the fulling bowls, a rotation can additionally take place by means of a gliding motion drive 4g of the fulling bowl carrier 4h of the base bowl 4a as shown, for example, in these diagrams, while this possesses e.g. the function of a pivoting motion device 11b and/or a rotation motion device 11c. This gliding motion could also be obtained by mounting the motion devices 11, 11a-11o on a housing which is then rotated.

FIG. 2 shows an example of a fulling device in which three motion devices 11, 11a-11o act in a fulling plane 22, which progresses vertically in relation to the plane of the edges of the fulling bowls 4 and through the middle point of the fulling bowls 4 or the joint replacement 3e. Two of these motion devices 11,11a-11o act in the same direction—but at a different height—on the fulling bowl 4/joint bowl 4b, and one acts vertically on the peak of the joint bowl 4b. The motion devices 11, 11a-11o in this example consist of motion devices 11e which act into both directions. In order to prevent a deviation of the motion from the fulling plane 22 of the fulling bowl 4/joint bowl 4b, it is fastened to a guide rail 21 which is guided within a slot, and which can glide in the slot of a guiding body 20 with linear and slot guides in the fulling plane 22.

FIG. 3 largely corresponds to FIG. 2, apart from the fact that the diagram consists of a section in the fulling plane 22 and two additional motion devices 11, 11a-11d, 11f-11o are mounted here. The additional motion devices 11, 11a-11d, 11f-11o are required here due to the fact that these motion devices consist of motion devices 11d, which only act into one direction. FIG. 3 can also (contrary to FIG. 2) be regarded as a fulling device for a hinge joint, i.e. a sectional depiction crosswise to the axis line of the hinge joint. A pivoting motion/rotation by the pivoting motion device 11b or the rotation motion device 11c of the fulling bowl carrier 4h of the base bowl 4a is then, however, excluded. The gliding motion drive 4g must then pivot the fulling bowl carrier 4h with the base bowl 4a into the axis direction of the hinge joint.

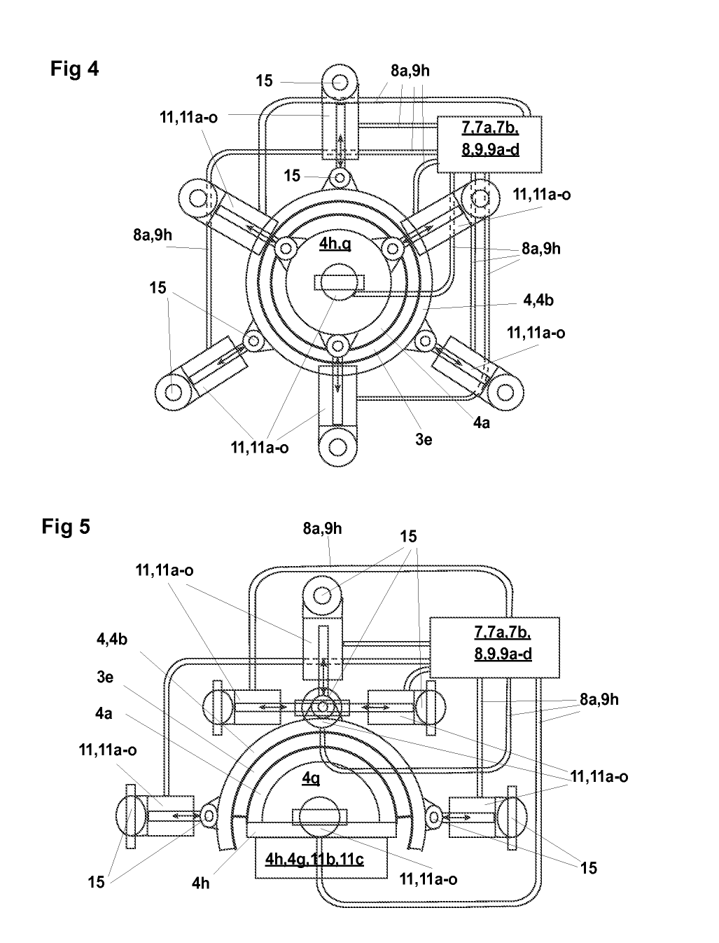

FIG. 4 and FIG. 5 show two further examples in which, however, there is no guide rail 21 which is guided in a slot, but fulling is performed in all three dimensions by motion devices 11, 11a-11o. FIG. 4 shows motion devices 11, 11a-11o which are mounted below the peak of the fulling bowl 4, and in FIG. 5, on a joint above the peak, on which the motion device 11, 11a-11o which acts on the peak is also mounted.

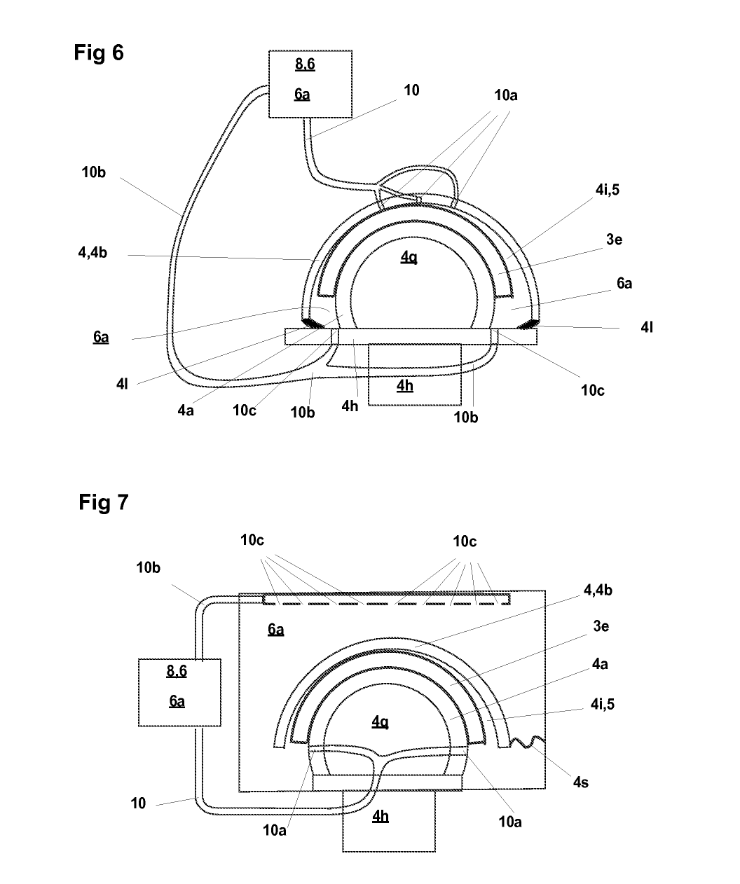

FIG. 6-FIG. 9 show examples of the components of the fulling device (for differentiation and shaping fulling processes) which contribute to the introduction (e.g. by means of diffusion) of joint replacement production substances 6a into the joint replacements 3e, particularly by flushing them into the fulling gap 4i, which may also have the function of a tissue cultivation chamber 5 (for the shaping fulling process). The flushing device 6 can be controlled by a control device 8, whereby the control device 8 of the flushing device 6 may be separated from the control device 8 of the fulling perform device 7, or may form one unit with it which controls both the fulling perform device 7 and the flushing process.

From the flushing device 6, the joint replacement production substances 6a flow through infeed lines 10 and via inflow openings 10a—directly or indirectly into the fulling gap 4i—and flow back into the flushing device 6 via return flow openings 10c and return flow lines 10b. The fulling bowls 4 and joint replacements 3e correspond to those in the preceding diagrams. In FIG. 6-FIG. 8, the fulling gap 4i also has the function of a tissue cultivation chamber 5, i.e. the joint replacement 3e can still be in its growth process, and is activated by the fulling process to do so.

The fulling gap 4i may also be at least partly sealed to improve the flushing process, as well as a part of the housing and the fulling process against its other part. The seals, which at least partly seal the fulling gap 4i and/or do so in at least one flow direction, consist of at least one of the following devices: a foil/membrane 4s, a flexible fulling gap sealing bar 4l, a fulling gap sealing tube 4r, an elastic connection/fulling gap edge foil 4k, a fulling gap foil pouch 4m, which fills out the fulling gap 4i, whereby parts of the fulling device, including the fulling gap edge seals 4j, consists of at least one of the following materials:

metal, foamed metal, hard and/or soft and/or elastic and/or compressible plastic, including polyactide, foam, foam rubber, ceramics, including tricalcium phosphate or tricalcium phosphate crystals, whereby at least one of the materials possesses at least one of the following attributes: permeable or impermeable to at least one of the joint replacement production substances 6a, firm, flexible, elastic, compressible, fluid absorbing, i.e. porous or spongious or perforated, and filled at least with one of the joint replacement production substances 6a-6h, with a gas and/or liquid.

FIG. 6 The joint replacement production substances 6a are introduced directly into the fulling gap 4i in this example, via inflow openings 10a in the joint bowl 4b. In this example, the fulling gap 4i is wholly or partly sealed by an elastic fulling gap sealing bar 4l, which glides on the fulling bowl carrier 4h of the base bowl 4a, i.e. the fulling gap sealing bar 4l can also be permeable to components of the joint replacement production substances 6a.

FIG. 7 At least one joint replacement substance 6a flows into the fulling gap 4i through inflow openings 10a, near the edge of the fulling gap 4i. The depth of the fulling gap increases slightly in the distance to the fulling point 4c or the fulling line 4d, so that joint replacement production substances 6a are suctioned into this area and distributed during the fulling process. The flow into the fulling gap 4i therefore takes place due to the fulling motion, i.e. due to the fact that the fulling gap 4i repeatedly opens and closes on one side, and—for example—also due to the fact that the joint bowl 4b is permeable to at least one component of the joint replacement production substances 6a, which are then also activated to penetrate into the fulling gap as a result of the fulling motion.

For this process, it can also be provided that the fulling bowl 4 (here joint bowl 4b) is repeatedly completely lifted off the joint replacement 3e at time intervals by the fulling perform device 7, so that the thus expanded fulling gap 4i fills with joint replacement production substances 6a. In all of these processes, the joint replacement production substances 6a are distributed throughout the fulling gap 4i by the fulling motion, and compressed against the joint replacement 3e, i.e. they are massively activated to diffuse into the joint replacement 3e. The pressure difference between the infeed 10 and the return flow line 10b causes the joint replacement 3e to be supplied. If the fulling bowl 4 is permeable, a foil/membrane 4s may also be mounted as shown on the right-hand edge of the joint bowl 4b, separating one part of the housing and the fulling device from the other part.

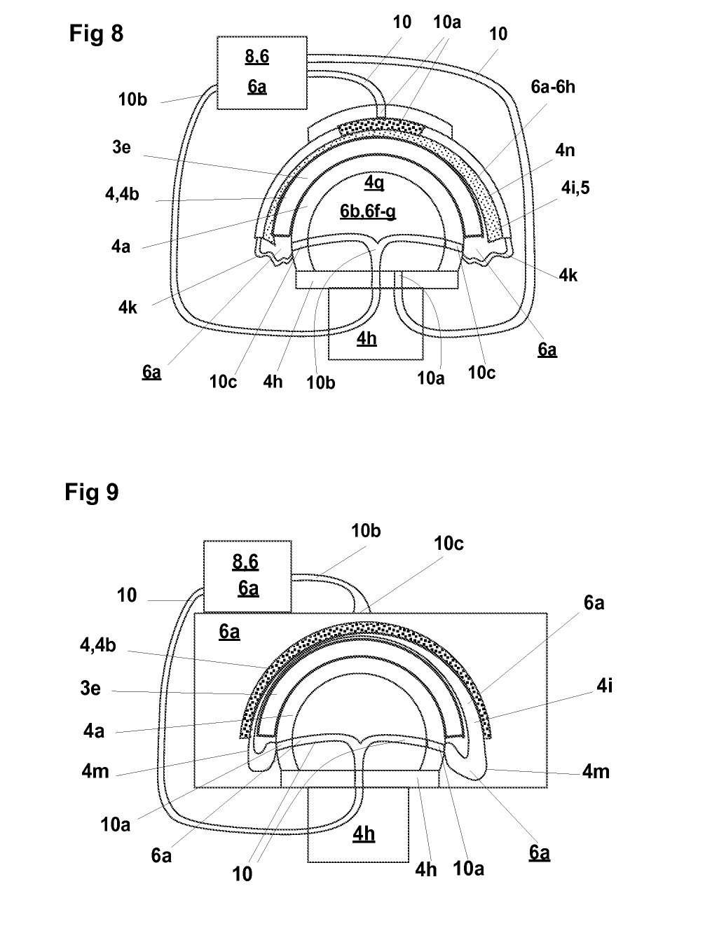

FIG. 8 This example consists of two joint replacement production substances 6a which are composed of different components. One of them is introduced into the joint gap 4i in this example via an inflow opening 10a in the center of the fulling bowl 4 (here joint bowl 4b), whereby this inflow opening 10a consists of an area which is permeable for the joint replacement production substances 6a. In this example, the fulling gap 4i is equipped with an absorbent fulling gap mat 4n. Joint replacement production substances 6a, which consist of other components, are introduced into the hollow space 4q of the base bowl 4a via an infeed 10. The base bowl 4a is thereby permeable to these substances. Here, this part of the joint replacement production substances 6a supplies the part of the joint replacement 3e which is mounted on the base bowl 4a, and then also flows into the fulling gap 4i with the fulling gap mat 4n. Here, the fulling gap 4i is sealed by a fulling gap edge seal 4j in the version of a fulling gap edge foil 4k.

FIG. 9 shows an example in which the fulling gap 4i is equipped with a fulling gap foil pouch 4m, which is permeable to the at least one joint replacement production substance 6a on at least one side within the fulling gap 4i. The fulling bowl 4/joint bowl 4b and/or the base bowl 4a can also be permeable to these substances. Outside the fulling gap 4i, the fulling gap foil pouch 4m is preferably impermeable to these substances, and possesses inflow opening 10a there to allow the joint replacement production substances 6a to flow in.

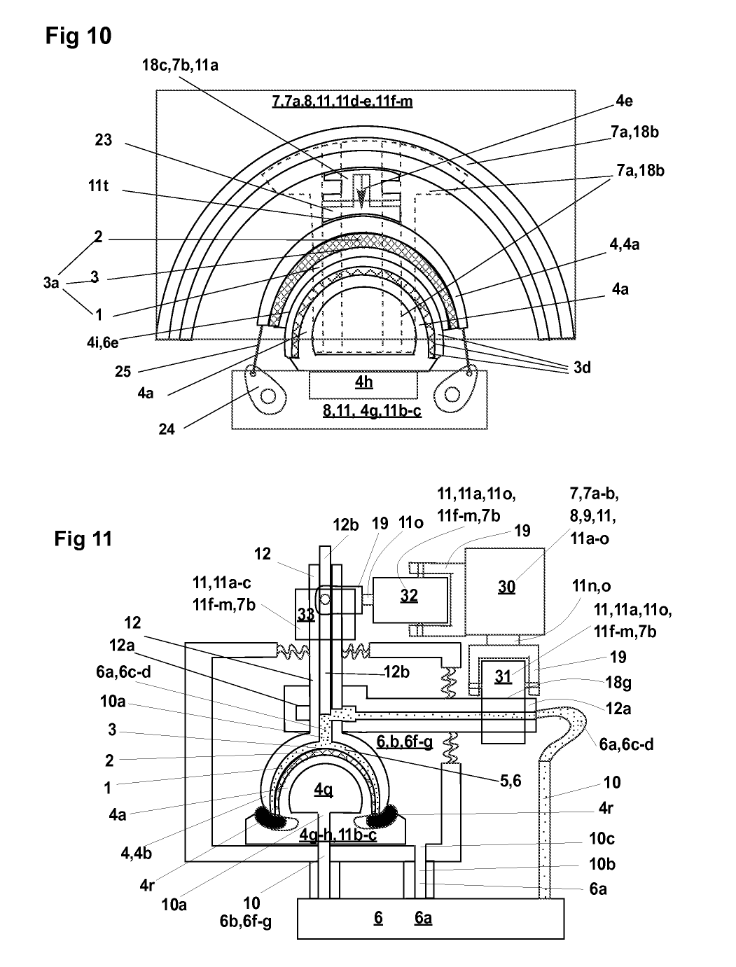

FIG. 10 is a section through an example of a fulling perform device 7 in which both fulling bowls 4 are respectively provided with a complete joint replacement 3a (i.e. a cartilage layer 1, carrying layer 2 and connective layer 3), i.e. the fulling process is performed from one joint replacement 3a to the opposite joint replacement 3d—and they show examples of a fulling motion device 7a and a fulling intensity device 7b. The fulling motion device 7a consists of two semicircular tracks 18b, i.e. semicircular gliding rails which are mounted in two planes which lie vertically above each other. The semicircular track 18b, which progresses crosswise in relation to the section of the image, is shown with dashed lines to avoid disturbing the overview of the other components. This semicircular track 18b is mounted on the other semicircular track 18b. A gliding sled 18c which possesses a fulling intensity device 7b with a linear motion device 11a glides on it; the linear motion device compresses a linear guided pressure body 23 under fulling pressure in a longitudinal direction; the pressure body transmits the fulling pressure to the fulling bowl 4, 4a via a pressure transmission gliding surface 11t. The motion of the second semicircular track 18b on the first, as well as the motion of the gliding sled 18c with the fulling intensity device 7b on the second semicircular track 18b, is performed by a motion device 11, which can act in a direction 11d or in opposite directions 11e, and whose drive can be powered by other motion devices 11f-11m.

Both fulling bowls 4 are base bowls 4a, i.e. fulling bowls on which the carrying layer 2 or the part of the joint replacement 3e opposing the joint gliding surface is mounted, i.e. one of the base bowls 4a bears a joint pan surface replacement 3a, and the other bears the opposing joint replacement 3d, in this case the joint head surface replacement. In this example, both joint replacements consist of three layers, i.e. the cartilage layer 1, carrying layer 2 and connective layer 3. The joint gap 4i is minimized in the entire area here, i.e. the fulling gap width consists only of a thin layer of a substance which also possesses joint lubricant attributes.

The joint pan surface replacement 3a, via the fulling bowl 4 of which the fulling pressure of the pressure transmission gliding surface 11t is applied, possesses a smaller spherical bowl section than the opposing joint head surface replacement 3d.

For this reason, the fulling bowl 4, 4a in the sectional plane of the diagram is connected with a traction connector 25 by two, although actually three or four pivoting levers 24; the traction connector is also moved by motion devices 11 and controlled by a control device 8—namely in such a manner that the joint pan surface replacement 3a moves across the entire opposing joint head surface replacement 3d, whereby this motion is coordinated with the motions of the fulling motion devices 7a by the control device 8.

As in the previous diagrams, rotation of the joint head basebowl 4a and its fulling bowl carrier 4h by a gliding motion drive 4g with motion devices 11b, c is also possible here. If this embodiment is used as a fulling device for a hinge joint, i.e. it is a sectional depiction crosswise to the axis line of the hinge joint, one of the semicircular tracks 18b is omitted, as well as the pivoting motion/rotation by the gliding motion drive 4g.

FIG. 11 shows an example of a fulling device which causes introduced tissue framework substances/particles 6d and cells 6c to combine and activate the formation of the tissues which are being produced (cartilage layer 1, carrying layer 2 and possibly a connective layer 3), i.e. it also specifically possesses the function of a shaping fulling process. For this purpose, the flushing device 6 introduces cells 6c, tissue particles (framework particles) 6d and tissue production substances 6b, 6e-6h via infeed 10—in this example, via a fulling bowl motion device with channel 12a and a fulling bowl holder with canal 12—into a tissue cultivation chamber 5(=fulling gasp 4i), which here possesses the shape of the cartilage layer 1 which is being produced. The fulling motion of the fulling bowl 4, which here consists of a joint bowl 4b, takes place nearly without fulling pressure at the outset—but rather only with a fulling depth—and thereby causes the coordinated integration of cells into the tissue structure, whereby the cells initiate the differentiation of the tissues into weight bearing cartilage due to this fulling process.

Here, the base bowl 4a may be additionally (see below) pivoted or rotated by means of gliding motions (via a gliding motion drive 4g) in order to encourage the even distribution of the tissue particles 6d or cells 6c. In this manner, it is possible to introduce tissue particles 6d and cells 6c continuously or in stages, which are gently pressed against the already existing tissue layer by the fulling motion, and are activated to connect with the tissue layer.

The firmer the cartilage tissue becomes, the more the fulling pressure 4e is increased by the control device 8, whereby the fulling depth 4f is pulled back insofar as necessary.

In this example, a second infeed line 10 is provided from the flushing device 6 with joint replacement production substances 6b, 6f-6g (but without tissue particles 6d and cells 6c), which are introduced into the hollow space 4q of the base bowl 4a. The base bowl 4a is permeable to these substances 6b, 6f-6g, whereby e.g. the nutrient supply to the joint replacements is improved. Likewise, the joint bowl 4b is permeable here to these joint replacement production substances 6b, 6f-6g (but not for tissue particles 6d and cells 6c). (Alternatively, the joint bowl 4b could be impermeable, then the infeed line 10 into the hollow space 4q of the base bowl 4a would perform the function of a return flow line 10b).

In this example, the fulling bowl holder with channel 12 contains a closing rod 12b. This can also be used to control the inflow of the joint replacement production substances 6a-6h. In particular, however, when no further supply of cells 6c and tissue particles 6d is required, it can be advanced so far that the previous opening in the fulling bowls 4, 4b is closed, and the spherical surface shape of the fulling bowl 4, 4b also exists in this area.

The device complexes 30, 31 and 32 can contain the fulling perform device 7 with its components 7a, 7b, a control device 8, and—as needed—a pressure system 9 for pressure motion devices 11 or pressure-independent motion devices 11, and e.g. into the vertical direction, a force transmission device 11n with a force transmission rod 11o. This force transmission rod 11o, which proceeds in a vertical direction, moves a bearing fork 19, in which the device complex 31 with the motion devices 11, 11a, 11o, 11f-11m, 7b (e.g. also with a fulling intensity device function) is embedded, and which moves the fulling bowl motion device with channel 12a around its pivot axis as well as into its axial direction in a gliding bearing 18g. In the horizontal direction, and via a bearing fork 19, a device complex 32 with motion devices 11, 11a, 11o, 11f-11m is pivoted; it moves the device complex 33 with the motion devices 11, 11a-11c, 11f-11m, 7b via a force transmission rod 11o and a bearing fork 19. As a fulling intensity device 7b, this moves the fulling bowl holder with channel 12 via an axial motion, and can also perform its fulling motion by means of pivoting and/or rotation.

Overall, this results in the fulling process as described above.

In this example, the fulling gap is sealed by a fulling gap sealing tube 4r. If this embodiment is used as a fulling device for a hinge joint, i.e. it is a sectional depiction crosswise to the axis line of the hinge joint, at least one of the bearing forks, e.g. bearing fork 19, is omitted, as well as the gliding motion drive 4g.

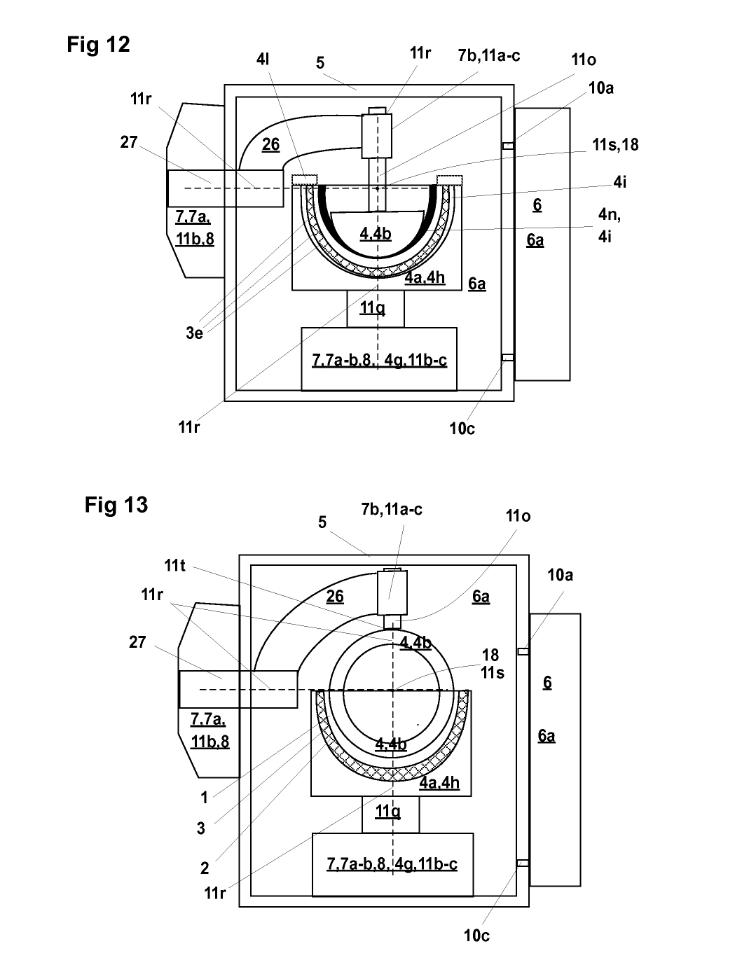

FIG. 12-14 show examples with mechanical motion devices of the fulling device 7, whereby its components—as shown in the preceding diagrams—can also be powered by other motion devices. The fulling bowls 4 are adapted to the shape of the joint replacement 3e. In a ball joint, this may lead to the following embodiments: a desired geometrical section of a spherical surface, e.g. an entire sphere; a sphere surface section; a sphere surface segment; the surface of a spherical ring; or also an asymmetrical spherical ring.

FIGS. 12 and 13 are examples of fulling motion devices 7 in which the actual fulling motion which is performed by the fulling bowl 4, 4b only takes place within one plane. Due to the rotation of the base bowl 4a by the gliding motion drive 4g, however, the fulling process takes place across the entire surface of the joint replacement 3e. The rotation thereby takes place via pivoting or rotation motion devices 11b-11c, namely via the axis 11q, on which the carrier 4h of the base bowl 4a is mounted. FIGS. 12 and 13 show a section through an example of a fulling perform device 7 with a fulling bowl 4, joint bowl 4b and a force transmission rod 11o in a fulling intensity device 7b, which consists of a linear motion device 11a to produce the fulling pressure, but may also include a pivoting motion device 11b or a rotation motion device 11c in order to pivot the fulling bowl in addition to the gliding motion device 4g or rotate it, i.e. to create an additional gliding motion between the fulling bowl 4, 4b and the joint replacement 3e.

These devices are mounted on the pivoting arm 26 of a pivoting shaft 27, whose axis line 11r progresses through the sphere center of the base bowl 4a, i.e. the joint center point 18 of the joint replacement 3e. The axis lines of the fulling intensity device 7b, 11a-11c and the axis of the gliding motion drive 4g, 11b-11c also progress through the axis intersection point 11s. The axis 11r of the pivoting shaft 27 and the fulling intensity device 7b are perpendicular to each other. The pivoting drive of the pivoting shaft 27 and the axis 11q of the fulling bowl carrier 4h are moved by the fulling perform device 7 with the fulling motion devices 7a, here the pivoting motion devices 11b, whereby these motions are coordinated by the control device 8. These fulling devices are also suitable for a hinge joint, i.e. they can be seen as a sectional depiction crosswise to the axis line of the hinge joint. A pivoting motion/rotation by the gliding motion drive 4g, 11b-11c and the pivoting/rotation motion devices in 7b, 11a-11c is then excluded.

In FIG. 12, the fulling bown 4, 4b is shaped approximately like a hemisphere (the joint replacement 1-3 to be fulled is a ball joint pan). Here, the fulling gap 4i contains a fulling gap mat 4n. In FIG. 13, the fulling bowl 4, 4b is shaped approximately like a spherical bowl. The force transmission rod 11o transmits the fulling pressure force to the fulling bowl sphere 4, 4b via a pressure transmission gliding surface 11t.

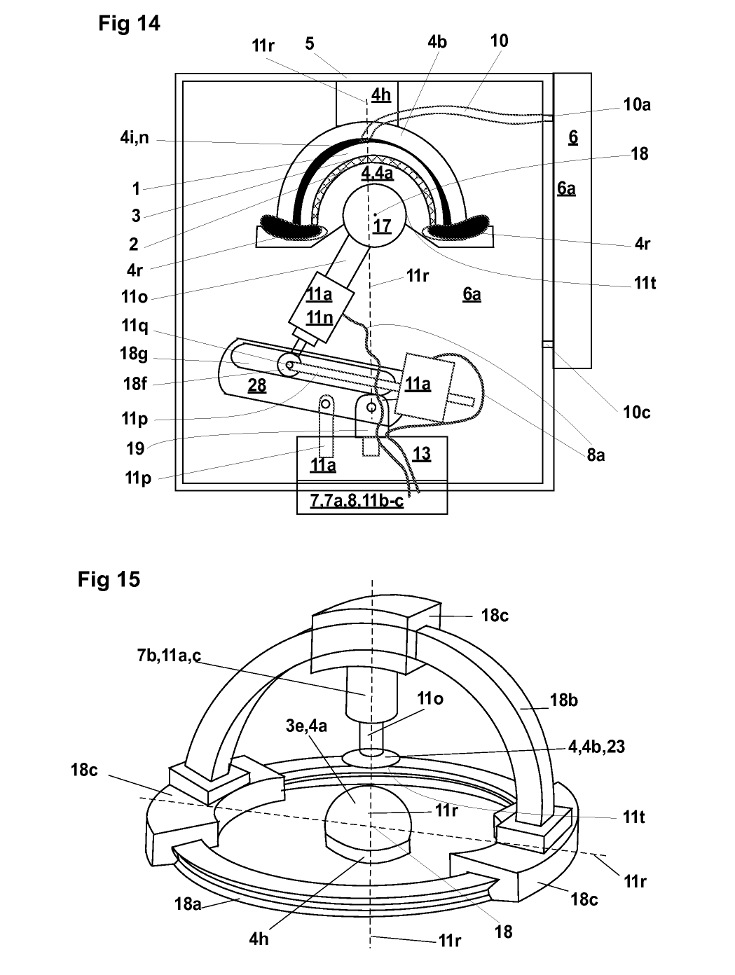

FIG. 14 shows an example of a fulling device in which the base bowl 4a performs the fulling process as the fulling bowl 4. For this, the base bowl 4a contains a ball joint in which a sphere 17 with a pressure transmission gliding surface 11t is positioned. The fulling pressure 4e is applied to this via a force transmission bar 11o. The fulling pressure is formed by the linear motion device 11a, which possesses the function of a force transmission device 11n, and in which the force transmission rod 11o is positioned. This motion device 11a, 11n is positioned in a manner which allows it to shift longitudinally via an axis 11q on a wheel 18f in a linear bearing 18g of a carrier rail 28. A forward drive rod 11p, which is powered by one of the linear motion device [sic: devices] 11a mounted on the carrier rail 28, adjusts the angle at which the force transmission rod 11o acts on the sphere 17 and thereby on the fulling bowl 4, 4a. The carrier rail 28 is positioned in a bearing fork 19 on a rotation body with axis bearings crosswise to the rotation axis 13, whose rotation axis 11 r runs through the center of the fulling bowl holder 4h and through the center point of the fulling bowl 4(=base bowl 4b). The pivoting and/or rotation motion of the rotation body 13 is generated by the pivoting motion device 11b and/or the rotation motion device 11c, and controlled by the control device 8. The angle of the carrier rail 28 to the horizontal plane is constantly altered by a forward drive rod 11p in the sense of the fulling motion.

The forward drive rod 11p is positioned on bearings on the carrier rail 28 for this purpose. The motion of the forward drive rod 11p is controlled by the fulling perform device 7—in this case, a fulling motion device 7a—which moves the forward drive rod 11p by means of a linear motion device 11a. In this manner, the fulling point moves nearly across the entire surface of the fulling bowl 4, 4b.

The control of the motion device 11a, 11n, which applies the fulling pressure 4e to the force transmission rod 11o, and the linear motion device 11a on the carrier rail 28 is performed by the control device 8 via the control lines 8a. In this example, an opening is provided on the peak of the joint bowl 4b with an infeed 10 for joint replacement production substances 6a, which are flushed into the fulling gap 4i and the fulling gap mat 4n which is located there. The fulling gap 4i is sealed by a fulling gap sealing tube 4r.

FIG. 15 shows an example of a fulling perform device 7. The joint replacement 3e is positioned on the base fulling bowl and mounted on the fulling bowl carrier 4h. It is fulled by the fulling bowl 4 (here joint bowl 4b). The fulling bowl 4, 4b is subjected to the fulling pressure 4e by a force transmission rod 11o and this, in turn, by a linear motion device 11a, whereby the linear motion device 11a is a part of the fulling intensity device 7b, which also produces rotation of the fulling bowl 4, 4b as needed by means of a rotation motion device 11c, i.e. a gliding motion of the fulling bowl 4, 4b in relation to the surface of the joint replacement 3e. This fulling intensity device 7b with its components 11a, 11c is mounted on a gliding sled 18c, which glides and is moved along the semicircular track 18b. This semicircular track 18b is positioned on the full circle track 18a with two gliding sleds 18c, resulting in a plane of motion which is square to the motion plane of the semicircular track 18b. The track motions of the gliding sleds 18c are controlled by motion devices 11 and control devices 8 which are not shown in the diagram. The circle center point of the circular tracks matches the center point 18 of the joint replacement 4, 4b.

In another embodiment, the bowl, which was thus far a fulling bowl 4, 4b, can also consist of a linear guided pressure body 23, which applies its pressure via a pressure transmission gliding surface 11t on the fulling bowl 4a (which is the joint replacement 3e in the above description) as a fulling motion.

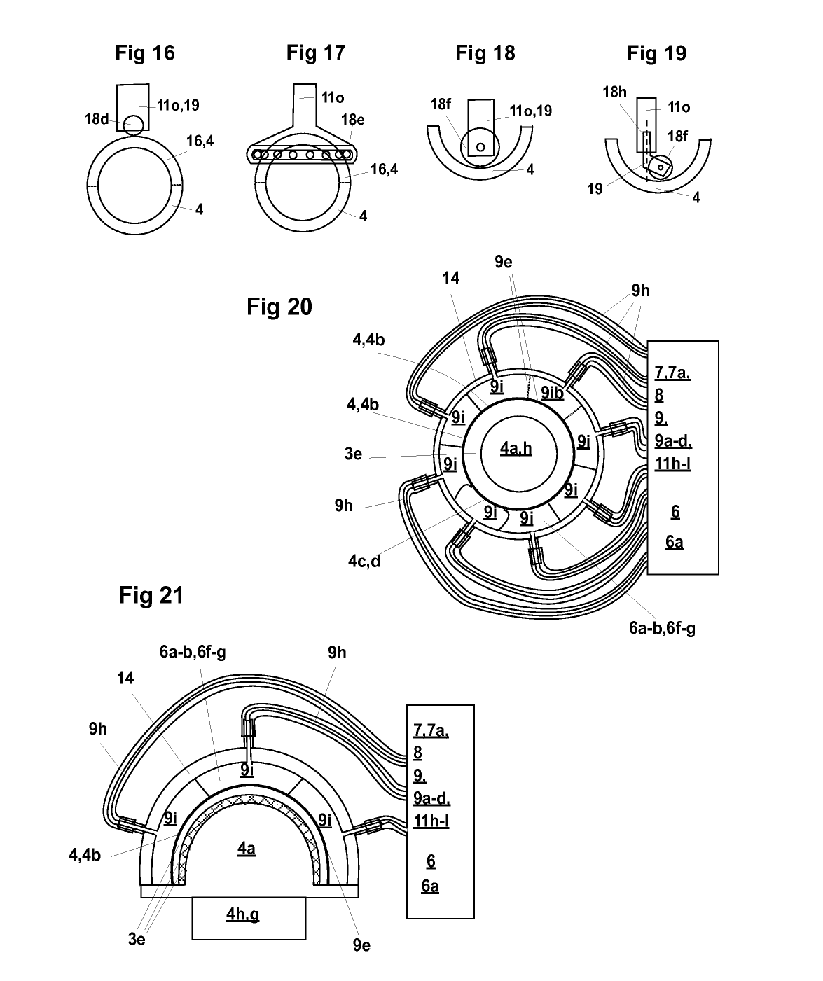

FIGS. 16-19 show examples of the fulling force transmission from a force transmission rod 11o to a fulling bowl 4, namely by means of rolling spheres 18d, a ball bearing ring 18e, and wheels/rollers 18f. This takes place e.g. via a bearing fork 19, whereby the bearing fork in FIG. 19 is positioned in a pivoting bearing 18h. In FIGS. 16 and 17 two embodiments of the fulling bowl 4 are shown, which can be a complete sphere 4 (and thereby full a joint pan replacement, for example), or a hemisphere 16, which is then suitable for fulling a joint head replacement, for example.

FIGS. 20 and 21 show a top view and cross-section of an example of a fulling device in which the fulling pressure 4e is transmitted to the fulling bowl 4, 4b via flexible pressure chambers 9i, or directly onto the joint replacement 3e. The pressure on the pressure chambers 9i is thereby generated from at least one pneumatic 9a and/or hydraulic 9b and/or hydropneumatic 9c and/or hydrodynamic 9d system. The pressure progression in the pressure chambers 9i is caused by a fulling motion device 7a, fulling intensity device 7b which is controlled by the control device 8. The pressure is transmitted to the pressure chambers 9i via the pressure lines 9h in such a manner that the maximum pressure moves across the surface of the fulling bowl 4 and/or the joint replacement 3e as fulling point 4c or fulling line 4d. On the side opposite the joint replacement 3e, the pressure of the pressure chambers 9i is received by a pressure chamber housing 14.

The pressure foils 9e which lie on the fulling bowl 4, 4b or the joint replacement 3e which is being formed are porous in this example, so that the pressure system 9 also has the function of the flushing device 6 with the joint replacement production substances 6a.

1. Fulling device for use in tissue engineering of a biological joint replacement to shape and/or differentiate a joint replacement which comprises at least one layer of a joint replacement, comprising a cartilage layer, carrying layer and connective layer between the carrying layer and the cartilage layer, the fulling device comprising:
two fulling bowls which form a fulling gap between them, in which there is a joint replacement and wherein the fulling bowls cover at least part of the surface of the joint replacement, and the shape and curvature of the fulling bowls, including a joint replacement which is mounted on them, at least nearly correspond to the surface of the fulled joint replacement,
a flushing device which introduces at least one joint replacement production substance into the fulling gap between the fulling bowls,
a fulling point or in hinge joints, a fulling line which form a pressure center and/or minimum distance between the fulling bowls, and which are recurrently moved/fulled with a fulling motion by at least one fulling bowl nearly across the entire surface of the joint replacement,
a fulling perform device, comprising:
a. at least one fulling motion device which acts on at least one fulling bowl and moves the fulling point or the fulling line, and thereby produces a fulling motion progression between the fulling bowls,
b. at least one fulling intensity device to adjust and control a fulling depth and/or fulling pressure from one of the fulling bowls against the other one, wherein the fulling depth corresponds to the reduction of the distance between the fulling bowls and the fulling pressure corresponds to the pressure force of one of the fulling bowls against the other one,
and at least one control device to control at least one of the devices comprising the fulling perform device, the fulling motion device, the fulling intensity device and the flushing device.

2. Fulling device according to claim 1, wherein the fulling motion device performs a fulling motion with at least one of the fulling bowls and the fulling bowls consist of comprise at least one of the following bowl combinations:
a base bowl on which the bone side of the joint replacement lies, and a joint bowl which acts on the joint surface side of the joint replacement,
two base bowls which each bear a joint replacement,
two fulling bowls between which material to produce a joint replacement, i.e. to shape it, is located.

3. Fulling device according to claim 1, wherein at least one of the fulling bowls is permeable to the at least one joint replacement production substance, in that it at least partly possesses at least openings or permeability to flush through at least one joint replacement production substance into and/or out of the fulling gap.

4. Fulling device according to claim 1, wherein the fulling gap for flushing/filling with at least one joint replacement production substance is at least partly closed by at least one fulling gap seal.

5. Fulling device according to claim 1, wherein the fulling gap possesses at least one of the following attributes:
a) a minimal fulling gap depth up to a depth which corresponds to a planned shape of the joint replacement, which corresponds to the depth and shape of a tissue cultivation chamber,
b) at least in the area of the fulling point or the fulling line, no gap depth or the minimum fulling gap depth, which slightly increases outside it,
c) a time-dependent, varying fulling gap depth which is caused by the fulling intensity device and the control device, with a repeating expansion of the fulling gap, i.e. a discontinuation of the fulling depth/the fulling pressure up to the removal of the fulling contact.

6. Fulling device according to claim 1, wherein the fulling motion device is formed so that the fulling bowls perform a gliding motion in relation to each other, in addition to the fulling motion.

7. Fulling device according to claim 1, wherein at least one of the following substances is provided for flushing in the fulling device, in addition to known joint replacement production substances:
a) a substance which is provided for the minimal gliding motion of the fulling process at a distance from the fulling point/the fulling line or for an additional fulling gliding motion, i.e. it acts as a joint lubricant and/or contributes to its formation,
b) a connective layer substance which, under the influence of the fulling process, produces a connective layer between one of the layers of the joint replacement or its connection to the bone which is provided to bear the joint replacement,
c) a differentiation substance which activates the differentiation of a joint replacement under the wandering fulling pressure, i.e. activates differentiation in the tissues due to the effects of the substance in combination with the fulling motion.

8. Fulling device according to claim 1, wherein an elastic mat which absorbs fluids is located in the fulling gap.

9. Fulling device according to claim 1, wherein the fulling gap contains a fulling gap foil pouch into which the flushing device introduces at least one joint replacement production substance, which fills the fulling gap and comprises elastic, flexible and/or porous material, and at least in the fulling gap is permeable to at least one joint replacement production substance.

10. Fulling device according to claim 1, wherein the fulling perform device possesses a pressure system in which pressure chambers are provided around at least one fulling bowl and are actively connected with it.

11. Fulling device according to claim 10, wherein the pressure system of the fulling perform device is simultaneously a flushing device, wherein a pressure medium of the pressure system simultaneously comprises the at least one joint replacement production substance as a flushing substance.
